SEQUENCE LISTING (1 sequence)

SEQ ID NO: 1 - protein, 135 aa, Monkeypox virus
RLNQCMSANKAAITDSAVAVAAASSTHRKVVSSTTQYDHKESCNGLYYQGSCYILHSDYKSFEDAKANCAAESSTLPNKSDVLTTWLIDYVEDTWGSDGNPITKTTSDYQDSDVSQEVRKYFCTAHHHHHHHHHH

(19) United States
(12) Patent Application Publication
(10) Pub. No.: US 2026/0035437 A1
(43) Pub. Date: Feb. 5, 2026

(54) IgY ANTIBODIES TARGETING MONKEYPOX VIRUS AND METHODS OF USE THEREOF

(71) Applicants: MARC (Medical Services and Scientific Research), Giza (EG); King Abdulaziz University, Jeddah (SA)

(72) Inventors: Sherif Ali El-Kafrawy, Jeddah (SA); Aymn Talaat Abbas, Jeddah (SA); Esam Ibraheem Azhar, Jeddah (SA); Laila Nabih Gadelrub Essa, Giza (EG); Suha Abdulaal Ali Farraj, Giza (EG)

(21) Appl. No.: 18/789,931

(22) Filed: Jul. 31, 2024

(51) Int. Cl. C07K 16/08; A61P 31/20

(52) U.S. Cl. CPC C07K 16/081; A61P 31/20; C07K 2317/11; C07K 2317/76

(57) ABSTRACT

Provided herein are methods of producing immunoglobulin Y (IgY) antibodies against monkeypox virus. The method includes immunizing hens with a peptide encoding at least one immunogenic domain of A29 fusion protein from the monkeypox virus and isolating the IgY antibodies against the A29 fusion protein from yolks of eggs laid by the immunized hens. Pharmaceutical compositions containing the IgY antibodies and methods of inhibiting or treating monkeypox virus infection are also provided.

### SEQUENCE LISTING

This document incorporates by reference an electronic sequence listing xml file, which was electronically submitted along with this document. The xml file is named 16410001AA_seglisting.xml, is 1842 bytes, and was created on Aug. 24, 2023.

### FIELD OF THE INVENTION

The invention generally relates to methods of producing a therapeutic composition of antibodies to treat or inhibit monkeypox virus (Mpox) infection. The invention further relates to methods of producing and using chicken egg yolk antibodies (IgY) to treat or inhibit infectious diseases, particularly Mpox.

### BACKGROUND

More than 86,700 cases were registered in the 2022-23 monkeypox (Mpox) outbreak until the end of March 2023 including 112 deaths as reported to the WHO from 110 countries. A significant majority of the cases were reported from nations where there is no history of Mpox transmission which urged the WHO to declare this outbreak as a global health emergency [1]. Mpox belongs to the orthopoxvirus (OPXV) genus along with cowpox, vaccinia, and smallpox viruses where all are double-stranded DNA viruses [2]. The name “monkeypox” comes from the fact that Mpox was first identified in macaque monkeys at a Danish research facility during a smallpox-like outbreak in 1958 [3]. In 1970, the first human Mpox case was reported in a child in the Democratic Republic of the Congo [4]. The Central African (Congo Basin) strains and the West African (WA) strains are the two primary clades of human Mpox that have been identified [5]. The two clades were renamed by WHO as Clade One (I) and Clade Two (II), respectively with two subclades for Clade II, Clade IIa, and Clade IIb. Currently, Clade IIb is linked to all sequences in the ongoing 2022-2023 Mpox outbreak [1]. For Mpox virus infections, there is currently no approved treatment. Nevertheless, antivirals that the United States Government has on hand and that were created for patients with smallpox may be helpful in treating Mpox. Tecovirimat, brincidofovir, and vaccinia immune globulin are the medical countermeasures that are currently on hand from the Strategic National Stockpile as possibilities for the treatment of Mpox. Cidofovir is also accessible commercially [6].

Passive immunity is achieved by receiving antibody treatments instead of producing them through the interaction between the antigen and the immune system, e.g. the maternal antibodies received by the fetus. The main advantage of this method is the ability to produce rapid protection rather than taking days to identify the antigen by the immune system and produce the corresponding antibody [7]. The egg yolk antibodies (IgYs), the isotype of mammalian IgG, are produced by injecting birds with a specific antigen that induces an immune response in the bird and generates IgYs which are then secreted in the egg yolk. Immunizing hens and harvesting the IgYs from the eggs provide a merciful and non-invasive approach to producing antigen-specific IgYs for use in passive immunity. IgYs also provide a more economical alternative to producing IgG antibodies in mammals since the quantity of IgY exceeds 18 times the production of the rabbits' IgG for the same purpose [8]. Although IgY has a similar structure to IgG, IgY has a higher molecular weight, 180 kDa versus 150 kDa for IgG, and the heavy chain of the IgY lacks the hinge region that exists in the IgG [9]. These differences give IgY special abilities to resist acidity, high temperatures, and hydrolysis. It can be taken for prevention or as well as for therapy to neutralize pathogens without interacting with the host immune system [10]. Additionally, on the production level, the activity of IgY antibodies is retained through different manufacturing steps and the lyophilized batches of IgY can be stored for several years [9]. This gives the IgYs their applicability as antiviral, antibacterial, and antiparasitic drugs [10].

A29 is a 14 kDa fusion protein in the surface membrane of the Mpox intracellular mature virus (IMV) [11]. When IMV binds to the cell, its membrane fuses with the plasma membrane and releases the virus's inner particle to the cytoplasm [12]. Mpox's A29 is homologous to the well-studied vaccinia A27 (VACV A27) protein with only four amino acid differences. Although one of these amino acids is in the required conserved sequence for heparin cell surface binding, both VACV A27 and Mpox A29 bind with similar efficiency to heparin. The binding with heparin is involved in the fusion from cell to cell. VACV A27 is also needed for intracellular enveloped virus formation in the Golgi bodies. In addition, it anchors the virus membrane during the formation of the inclusion bodies [13].

### SUMMARY

Embodiments of the disclosure provide in vitro and in vivo methods and compositions useful for neutralizing Mpox virus. The disclosure further comprises chicken egg yolk antibodies (IgY Abs) specific to the Mpox virus A29 fusion protein.

One aspect of the disclosure provides a method of producing IgY antibodies against Mpox virus, comprising immunizing hens with a peptide comprising at least one immunogenic domain of A29 fusion protein from the Mpox virus and isolating the IgY antibodies against the A29 fusion protein from yolks of eggs laid by the immunized hens. In some embodiments, the peptide has the amino acid identity of SEQ ID NO: 1:

RLNQCMSANKAAITDSAVAVAAASSTHRKVVSSTTQYDHKESCNGLYYQ
GSCYILHSDYKSFEDAKANCAAESSTLPNKSDVLTTWLIDYVEDTWGSD
GNPITKTTSDYQDSDVSQEVRKYFCTA

In some embodiments, the peptide has an amino acid sequence that is at least 80%, 85%, 90%, 95%, 96%, 97%, 98%, 99% identical to SEQ ID NO: 1.

In some embodiments, the peptide is a function-conservative variant of SEQ ID NO: 1. Accordingly, “function-conservative variants” are those in which a given amino acid residue in a protein has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” has the same or substantially similar properties or functions as the native or parent protein to which it is compared.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine.

Another aspect of the disclosure provides a pharmaceutical composition comprising IgY antibodies against Mpox virus, wherein the IgY antibodies are produced in eggs laid by hens immunized with a peptide comprising at least one immunogenic domain of A29 fusion protein from the Mpox virus. In some embodiments, the peptide has the amino acid sequence identity of SEQ ID NO:1.

Another aspect of the disclosure provides a method of inhibiting or treating a Mpox virus infection in a subject in need thereof, comprising isolating IgY antibodies to Mpox virus, wherein said IgY antibodies to Mpox virus are generated by immunizing hens with a peptide comprising at least one immunogenic domain of A29 fusion protein from the Mpox virus; preparing a pharmaceutically acceptable composition comprising the IgY antibodies; and administering a therapeutically effective amount of the pharmaceutically acceptable composition to the subject. In some embodiments, the peptide has the amino acid sequence identity of SEQ ID NO:1. In some embodiments, the composition is administered via a route of administration selected from the group consisting of intravenous injection, intravenous infusion, intraperitoneal injection, intraperitoneal infusion, intranasal, topical, and oral. In some embodiments, the therapeutically effective amount ranges from 0.1 to 1,000 mg/kg. In some embodiments, the subject is a human.

Other features and advantages of the present invention will be set forth in the description of invention that follows, and in part will be apparent from the description or may be learned by practice of the invention. The invention will be realized and attained by the compositions and methods particularly pointed out in the written description and claims hereof.

### BRIEF DESCRIPTION OF THE DRAWINGS

The accompanying drawings, which are incorporated in and constitute a part of this specification, illustrate embodiments of the invention and, together with a general description of the invention given above, and the detailed description given below, serve to explain the invention.

FIG. 1. The molecular weight and purity of anti-Mpox-A29 IgY antibodies. On the left, SDS-PAGE shows the two IgY chains (73 kDa, and 3 lkDa) using 10% resolving SDS-PAGE gel. On the right, a western blot shows the same chains using Rabbit Anti-Chicken IgY Antibody (HRP) conjugate.

FIG. 2. Kinetics of the response of the egg yolk IgY antibody to Mpox-A29 recombinant protein injected into hens immunized group (X) compared to non-immunized group (J). Each week is represented by a pool of egg yolks of individual chickens from each group.

FIGS. 3A-D. IgY antibodies. (A) The A29 protein of Mpox (Antigen) subjected to SDS PAGE (from supplier package insert). (B) The A29 protein of Mpox (Antigen) run in SDS-PAGE under the same experimental conditions as the IgY-Abs. Both show the molecular weight of the antigen (≈14 kDa). (C) Western blot analysis of the anti-A29 IgY antibody response from immunized hens (X) showing a band at 14 kDa indicating the existence of the anti-A29 IgY antibodies. (D) Western blot analysis of the IgY antibody response from non-immunized hens (J) showing no band at 14 kDa indicating no anti-A29 IgY antibodies in non-immunized hens (J).

### DETAILED DESCRIPTION

The following descriptions and examples illustrate some exemplary embodiments of the disclosed invention in detail. Those of the skill in the art will recognize that there are numerous variations and modifications of this invention that are encompassed by its scope. Accordingly, the description of a certain exemplary embodiment should not be deemed to limit the scope of the present invention.

Mpox virus is a species of double-stranded DNA virus that causes Mpox disease in humans and other mammals. Common symptoms of Mpox are a skin rash or mucosal lesions accompanied by fever, headache, muscle aches, back pain, low energy, and swollen lymph nodes. A29 is a 14 kDa fusion protein in the surface membrane of the intracellular mature virus. Embodiments of the disclosure provide in vitro and in vivo methods and compositions which incorporate anti-A29 IgY antibodies to neutralize the Mpox virus.

One embodiment of the disclosure provides a method of generating IgY Abs against A29 fusion protein by immunizing hens and isolating the IgY Abs from eggs laid by the hens. The immunogen may comprise a full-length A29 protein or a fragment thereof. In one embodiment, the immunogen is an A29 protein having the amino acid identity of SEQ ID NO:1.

In another embodiment, the Mpox-specific IgY is a treatment for Mpox infection in a subject. In another embodiment, the disclosure provides a method for using IgY Abs to inhibit and/or treat disease in a subject at risk for Mpox infection. In another embodiment, the disclosure provides in vitro methods of inhibiting Mpox spread or replication by contacting the virus with IgY antibodies as described herein for a time sufficient to inhibit viral spread or replication.

The method of producing IgY antibodies against Mpox virus, comprises the steps of immunizing hens with a peptide comprising at least one immunogenic domain of A29 fusion protein from Mpox virus, and isolating the IgY antibodies against the A29 fusion protein from yolks of eggs laid by the immunized hens. In one embodiment, the amount of immunogen used for immunizing hens is in the range of 10 to 1,000 μg. In one embodiment, the amount of immunogen administered to hens is in the range of 100 to 500 μg. In one embodiment, the amount of immunogen administered to hens is in the range of 150 to 250 μg. The immunogen may be administered on 1 to 5 or more occasions at about 1-3 week intervals, e.g. 2 week intervals. For example, the immunogen may be administered at about day 0, day 14, and day 28.

Another embodiment provides a pharmaceutical composition comprising IgY antibodies against Mpox virus, wherein the IgY antibodies are produced in eggs laid by hens immunized with a peptide comprising at least one immunogenic domain of A29 fusion protein from the Mpox virus. In one embodiment, the pharmaceutical composition comprises IgY antibodies against A29 protein having the amino acid sequence identity of SEQ ID NO:1.

In yet another embodiment, the invention is a method of inhibiting or treating a Mpox virus infection in a subject in need thereof, comprising the steps of isolating IgY antibodies to Mpox virus, wherein said IgY antibodies are generated by immunizing hens with a peptide comprising at least one immunogenic domain of A29 fusion protein from the Mpox virus; preparing a pharmaceutically acceptable composition comprising the IgY antibodies; and administering a therapeutically effective amount of the composition to the subject. The route of administration of the pharmaceutical composition may be selected from the group consisting of intravenous injection, intravenous infusion, intraperitoneal injection, intraperitoneal infusion, intranasal, topical, oral or any other route deemed appropriate by a practitioner.

By a “therapeutically effective amount” is meant a sufficient amount of active agent to treat the disease or disorder at a reasonable benefit/risk ratio applicable to any medical treatment. It will be understood that the total daily usage of the compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific active agent employed; and like factors well known in the medical arts. For example, it is well within the skill of the art to start doses of the compound at levels or frequencies lower than those required to achieve the desired therapeutic effect and to gradually increase the dosage or frequency until the desired effect is achieved. However, the daily dosage of the active agent may be varied over a wide range from 0.01 to 1,000 mg per adult per day. In particular, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, in particular, from 1 mg to about 100 mg of the active ingredient. The therapeutically effective amount may be in the range of 0.1 to 1,000 mg/kg. In some embodiments, the therapeutically effective amount is in the range of 1 to 500 mg/kg, 5 to 100 mg/kg, 10 to 50 mg/kg or 15 to 25 mg/kg.

A subject to be treated by methods described herein includes humans or other mammals or other non-human animals including, but not limited to primates, dogs, horses, cats, rabbits, gerbils, hamsters, rodents, birds, aquatic mammals, cattle, pigs, camelids, and other zoological animals.

As used herein, the terms “antigen” and “immunogen” are used interchangeably. “Antigen” typically designates an entity or epitope that is bound by an antibody and the entity or epitope that induces the production of the antibody. More current usage limits the meaning of antigen to that entity bound by an antibody, while the word “immunogen” is used for the entity that induces antibody production. Where an entity discussed herein is both immunogenic and antigenic, reference to it as either an immunogen or antigen will typically be made according to its intended utility.

The terms “peptide”, “polypeptide” and “protein” may be used interchangeably herein, although a protein is typically a linear sequence of about 100 or more amino acids covalently joined by peptide bonds, a polypeptide is typically a linear sequence of about 55 to about 100 amino acids covalently joined by peptide bonds and a peptide is typically a linear sequence of about 55 or fewer amino acids covalently joined by peptide bonds.

The methods of the invention provide certain advantages over conventional treatments. For example, they can be produced naturally, i.e., using the immune system of chickens, thus avoiding complications or side effects that might arise from synthetic drugs. The safety profile for IgY antibodies is well-established. The antibodies can be raised against multiple antigenic targets, and this can reduce the probability of viral resistance. Another advantage is that being specific to the target pathogen, they will not affect the host microbiome of a subject who receives the IgY Abs, thus avoiding undesirable side effects associated with depletion or imbalance of host microbial populations. Furthermore, IgY Abs are not known to deposit in the muscle tissue, thus the ability to fight infections in animals avoids the possible defilements of protocols in several countries that prohibit the use of antibiotics in livestock industry for production of meat products.

The methods of the invention provide a non-invasive approach and pain-free animal-friendly technique for the production of antibodies in animals, since immunized hens will provide a steady supply of eggs from which the IgY Abs may be extracted. Large-scale production of IgY Abs can be attained by one chicken (about 22 g/year), with 2-10% of the antibodies being specifically targeted. Another advantage is the convenience of storage of the eggs, including long term storage of IgY Abs in eggs for as much as a year or more at 4° C. prior to isolation of the IgY Abs. Furthermore, the existing infrastructure of chicken farms for the large-scale production of eggs reduces the barriers for scale up the production of IgY Abs to an industrial scale.

The ability to store eggs long-term and/or to scale up production is yet another advantage of the invention. In cases of new viral outbreaks, IgY Abs can be produced within a short period of time (6 weeks from vaccination of hens) and can be formulated to provide immediate defense to individuals and the environment such as schools, airplanes and hospitals. The eggs can be stored in large quantities for global use in the time of pandemic.

After isolation from eggs, IgYs can be extremely stable at pH 4-9 and in hot conditions up to 65 degree Celsius in aqueous conditions. In the presence of pepsin, they can retain antigen-binding activity at pH 4-6, which demonstrates that they are very appropriate candidates for most types of processing and applications. The sialic acid high content in IgYs increases the half-life of the isolated antibodies, as compared to antibodies with low sialic acid content (Liu 2015). Without being bound by theory, this property may contribute to IgY-based therapy due to a long circulating half-life that increases the efficacy against the infections.

Yet another advantage of the invention is that low antigen quantities are needed to get an efficient immune response in chicken compared to the mammals commonly used for raising antibodies. It is also noteworthy that the performance of the antibodies of the invention is more efficacious compared to mammalian IgG-Abs, since IgY Abs have better binding avidity to the targeted antigens. The evolutionary distance between mammals and birds gives the IgY Abs the further advantage of producing antibodies against conserved mammalian proteins more easily and successfully than producing IgG-Abs in other mammals. Compared to mammalian IgG-Abs, IgY Abs does not activate or interact with mammalian Fc receptors or fix mammalian complement components, thus limiting the potential for inflammatory or dangerous immune responses in the subject receiving the IgY Abs treatment. Furthermore, there is no cross-reactivity with rheumatoid factors. For passive immunotherapy the IgY antibodies used can be given to a wide range of individuals belonging to any age and immunodeficient patients and pregnant women can be included.

The present disclosure also provides compositions for use in eliciting an immune response. The compositions may be utilized to immunize hens to produce IgY antibodies that prevent or treat Mpox infection in mammals. By eliciting an immune response, we mean that administration of the antigen causes the synthesis of specific antibodies. The compositions include one or more isolated and substantially purified peptide or polypeptide as described herein, and a pharmacologically suitable carrier. The polypeptide(s) or protein(s) may be the same or different, i.e., the composition may be a “cocktail”, or a composition containing only a single type of peptide or protein. The preparation of such compositions for use as immunogens is well known to those of skill in the art.

Pharmaceutical compositions comprising the anti-A29 IgY Abs are prepared either as liquid solutions or suspensions, however solid forms such as tablets, pills, powders and the like are also contemplated. Solid forms suitable for solution in, or suspension in, liquids prior to administration may also be prepared. The preparation may also be emulsified. The active ingredients may be mixed with excipients which are pharmaceutically acceptable and compatible with the active ingredients. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. The pharmaceutical compositions of the present invention may be administered as an oral form, wherein various thickeners, flavorings, diluents, emulsifiers, dispersing aids or binders and the like may be added. The composition of the present invention may contain any such additional ingredients so as to provide the composition in a form suitable for administration. The final amount of IgY Abs in the formulations may vary. However, in general, the amount in the formulations will be from about 0.01-99%, weight/volume. The amount of antibodies administered to a subject may range from 100 μg to 100,000 mg.

The methods involve administering a pharmaceutical composition comprising anti-A29 IgY Abs in a pharmacologically acceptable carrier to a mammal. The mammal may be a human, but this need not always be the case, as veterinary applications of this technology are also contemplated. Other species include but are not limited to companion “pets” such as dogs, cats, etc.; food source, work and recreational animals such as cattle, horses, oxen, sheep, pigs, goats, and the like; or even wild animals that may be found to serve as a reservoir of Mpox. The pharmaceutical compositions of the present invention may be administered by any of the many suitable means which are well known to those of skill in the art, including but not limited to by injection, inhalation, topically, orally, intranasally, by ingestion of a food product containing the anti-A29 IgY Abs, etc. The mode of administration injection may be subcutaneous, intramuscular, intravenous, or intraperitoneal. In addition, the compositions may be administered in conjunction with other treatment modalities such as substances that boost the immune system, various anti-bacterial, chemotherapeutic agents, antibiotics, and the like.

Some examples of materials which can serve as pharmaceutically acceptable carriers include, but are not limited to, ion exchangers, alumina, aluminum stearate, lecithin, serum proteins (such as human serum albumin), buffer substances (such as Tween® 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene[1]polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other nontoxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The present disclosure also encompasses antibodies to the epitopes and/or to the polypeptides disclosed herein. Such antibodies may be polyclonal, monoclonal or chimeric that are generated in chickens/eggs.

Before exemplary embodiments of the present invention are described in greater detail, it is to be understood that this invention is not limited to any particular embodiments described herein and may vary. It is also to be understood that the terminology used herein is for the purpose of describing particular embodiments only, and is not intended to be limiting, since the scope of the present invention will be limited only by the appended claims.

Where a range of values is provided, it is understood that each intervening value between the upper and lower limit of that range (to a tenth of the unit of the lower limit) is included in the range and encompassed within the invention, unless the context or description clearly dictates otherwise. In addition, smaller ranges between any two values in the range are encompassed, unless the context or description clearly indicates otherwise.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Example

### Summary

Monkeypox is a rare disease caused by the monkeypox virus (Mpox), a member of the Orthopoxvirus family. At the end of July 2022, World Health Organization (WHO) declared Mpox outbreak a global health emergency. We generated chicken IgYs specific to the Mpox's A29 protein by immunizing egg-laying chickens with recombinant A29 protein. Eggs were collected daily from immunized chicken; yolks were separated and pooled weekly and IgYs were extracted from the pooled egg yolks. IgY titers were shown to rise to a titer of 102400 using ELISA up to week 8 followed by a decline down to 12800 at week 13. Western blotting assay demonstrated specific binding of the generated IgY antibodies to the Mpox A29 protein.

### Materials and Methods

### Immunization of Laying Hens

Six Lohmann laying hens (18 weeks old) were used for egg production. Each hen was placed individually in a chicken cage in a light-dark cycle of 16 hours and 8 hours, respectively, at room temperature (24±3° C.) and a humidity of 30% to 40%.

When all the hens started laying eggs (at 22 weeks of age), the eggs were collected for a week (week-1) to be used as a preimmunization control (C). Hens were divided into two groups (3 hens each), the immunized group (X) received a freshly prepared emulsion of 200 μg the recombinant Mpox A29 protein (40891-V08E, Sino Biological Inc, China) in a 1:1 ratio with Freund's Complete Adjuvant (F5881, Sigma, USA) for initial immunization, and Freund's Incomplete Adjuvant (F5506, Sigma, USA) for subsequent booster doses. The second group (J) received only Freund's Complete Adjuvant for initial immunization and Freund's Incomplete Adjuvant for subsequent booster doses which were both mixed with water to adjust the volume to a 1:1 ratio as for the immunized group. Both groups were injected on day 0, day 14, and day 28. Each dose was administered in four sites at the left and right sides of the pectoral muscle. Eggs were collected daily after immunization and stored at 4° C. until the time of IgY isolation from the yolk.

### Isolation and Purification of Yolk IgY

Egg yolks of each week from both study groups (X and J) were pooled after being separated from the egg whites. Fifteen ml of the pooled yolk was used as a starter material for IgY purification following the polyethylene glycol (PEG) precipitation procedure [14]. Briefly, the yolk of each pool was diluted by mixing with twice the egg yolk volume of Phosphate-buffered saline (PBS) (ML116, Hi-Media, India) and 3.5% PEG 6000 (05312, Loba, India) of the total volume and centrifuged at 12000 rpm for 20 mins; after mixing and centrifugation the components were separated into two layers. The solids and fatty precipitate layer were discarded, and the supernatant layer containing the IgY with other proteins was filtered. The supernatant was mixed with 8.5% of PEG 6000, the mixture was centrifuged at 12000 rpm for 20 mins, and the supernatant was discarded then the precipitate was dissolved in 10 ml of PBS followed by the addition of 12% PEG 6000 and centrifugation at 12000 rpm for 20 mins. The final precipitate was dissolved in PBS 2 ml, and the concentration and the purity of the extracted IgY were measured using NanoDrop OneC (ND-ONEC-W, Thermo Fisher, USA) using the absorbance at 280 (A280) nm and A260/A280, respectively, according to the manufacturer's instructions.

### SDS-PAGE Assay

To determine the molecular weight of IgY and antigen, 4.3 μg from the extracted IgY were mixed with 2× sample buffer [5% mercaptoethanol (92441, Advent, India), 2% sodium dodecyl sulfate (90552, Advent, India), 10% glycerol (IG005, Piochem, Egypt), 0.5 M Tris (pH 6.8)] and boiled for 10 min at 100° C. Sodium dodecyl sulfate-polyacrylamide gel (SDS-PAGE) (10%) was prepared following TGX Stain-Free™ FastCast™ Acrylamide Starter Kit (1610182, BioRad, USA). The prepared sample was loaded into the well and five pls of 3 color Prestained Protein Ladder (PG-PMT2922, Genetix, India) was loaded as a molecular weight marker. After filling the electrophoresis tank with 1× of the running buffer from 10× Tris/Tricine/SDS Running Buffer (1610744, BioRad, USA), the run was performed at 200 volts for one hour using a Mini-PROTEAN® Tetra Vertical electrophoresis cell (1658004, Bio-Rad Laboratories, USA). The gel was then soaked in prepared 0.1% Coomassie® blue dye (0294100025, Loba, India) for 45 min to visualize the bands then soaked in a De-Stain buffer (20% methanol (1.06007.4000, Merck, Germany) and 10% acetic acid (SA0010102500, SUVCHEM, India) until the bands became clear. The molecular weight and purity of the bands were analyzed using GeneTools® software version 4.3.14 (Syngene, England) on the pictures taken by gel documentary Nugenius® (Syngene, England).

### Western Blotting Assay

To check the specificity of the anti-Mpox-A29 IgY antibodies, western blotting was done using 500 ng of recombinant A29 protein in 12.5 μL mixed with 2× sample buffer and loaded onto the SDS-PAGE gel alongside the molecular weight marker as previously described. Immuno-blot polyvinylidene fluoride (PVDF) membrane (1620177, Bio-Rad Laboratories, USA) was activated for 15 seconds in methanol. PVDF membrane and filter papers were incubated in 1× transfer buffer (192 mM Glycine, 25 mM Tris, 10% Methanol) for 3 min. Using Trans-Blot® Turbo™ Transfer System (1704150, Bio-Rad Laboratories, USA), the samples in the run gel were electrically transferred under 30 volts for 5 min. The PVDF membrane was washed for 5 min with Tris-buffered saline (50 mM Tris-Cl and 150 mM NaCl (1.06404.1000, Merck, Germany)) then blocked with blocking buffer consisting of Tris-buffered saline with 0.1% Tween® 20 (TBS-T) detergent and 5% w/v skimmed milk for 1 h at room temperature. The membrane was washed for 10 min three times in wash buffer (TBS-T) and cut into strips each had two lanes, one had the molecular weight and the other had the recombinant A29 antigen. Each strip was incubated in a 1:100 dilution of the extracted IgY from the X (immunized) or J (non-immunized) groups. After incubation, the strips were washed again for 10 min three times in the wash buffer. Then, the strips were incubated in a 1:25,000 dilution of Rabbit Anti-Chicken IgY Antibody (HRP) conjugate (12-341, Sigma-Aldrich, Germany) in a blocking buffer for 1 h at room temperature. The strips were washed for the last time three times for 10 min in the wash buffer. Finally, the strips were incubated with 1 Component TMB Membrane Peroxidase Substrate for western blotting (50-77-18, KPL company, USA) for 10 min at room temperature and the reaction was stopped by rinsing with distilled water and the strips were photographed. Following the previously mentioned procedure, 4.3 μg from the extracted IgY was run, blotted and visualized. As in SDS-PAGE Assay, the pictures were taken by the gel documentary Nugenius® and the molecular weight calculation was done using GeneTools® software.

### ELISA Assay

The titer of the generated anti-Mpox-A29 IgY (antibodies) was determined by enzyme-linked immunosorbent assays (ELISA). Briefly, microtiter plates were coated with 500 ng/mL antigen in PBS by adding 100 μL/well and incubated overnight at 4° C. The plate was washed twice with 0.05% Tween®-20 (6417D, Loba, India) in PBS (PBS-Tween) using Microplate Washer model 50TS (Biotek, USA). the wells were then blocked with 200 μLs of blocking buffer consisting of 5% skimmed milk (0230, Biobasic, Canda) in PBS-Tween at room temperature for one hour. The wells were then washed three times with PBS-Tween®.

Starting with a 1:50 dilution of the purified IgYs, a 2-fold serial dilution from all study groups (X, J, and preimmunization eggs) was done in blocking buffer and 100 μLs were added to each well. Plates were then incubated at 37° C. for one hour, the plates were washed five times with PBS-Tween®. At a dilution of 1:25,000, 100 μL of Rabbit Anti-Chicken IgY Antibody, horseradish peroxidase (HRP) conjugate (12-341, Sigma-Aldrich, Germany) was added to each well and incubated for one hour at 37° C. The plates were then washed five times, then 100 μLs of 3,3′,5,5′-Tetramethylbenzidine (TMB) ELISA substrate (TM001-B100ML, Bio-Helix, Taiwan) was added to each well and incubated for 30 mins for color development. This reaction was stopped by adding 30 μl of 1N HCL (89201, brand chemicals, Egypt) to each well. The optical density (OD) of each well was read at both 450 nm and 620 nm, using a microplate reader model 800TSUV (Biotek, USA). PBS was used as a blank, and the maximum dilution of the sample that resulted in an OD value 2.1 times higher than the preimmunization control reflects the titer of anti-Mpox-A29 IgY.

### Cytotoxicity Assay

The cytotoxicity assay was performed using the 3-(4,5-dimethylthiazol-2-yl)-2,5-diphenyltetrazolium bromide (MTT) (Roche, Germany) protocols. Vero E6 cell monolayers with 4×10³ cells/mL plated onto 96-well culture plates were washed 3 times with phosphate buffer saline (PBS) 1× pH 7.4. 100 μLs of prepared working solution of IgY antibodies were added into each well of the 96-well plate and another 100 μLs of maintenance medium were added onto the wells (3 wells per each dilution in two independent experiments). Negative control cells were incubated with 100 μLs of water instead of the IgY solution. The cells were incubated at 37° C. for 48 h in a 5% CO₂ atmosphere, the supernatant was then aspirated, and cells were washed 3 times with PBS, then 20 μL of MTT solution was added to each well and incubated for 4 h at 37° C. Solubilization solution was added and incubated overnight at 37° C. The plate was read using an ELISA reader (Synergy 2 microplate, BIOTIK, South Korea) with a reference wavelength of 570 nm (OD₅₇₀).

Cytotoxicity was calculated from mean values according to the following equation: (1—(OD₅₇₀ drug/OD₅₇₀ control)×100). Cytotoxicity graphs were then generated by plotting the percentage of cytotoxicity versus log₁₀ the drug concentration using Graphpad® prism 9 (Version 9.0.0) and the CC₅₀ was calculated using the nonlinear curve fitting with a variable slope where the equation of the fit curve is:

Y=100/(1+10\hat{ }((LogCC₅₀−X)*HillSlope))

where Y is the % cytotoxicity and X is the concentration.

### Plaque Reduction Neutralization Assay

Live virus experiments were performed in a biosafety level 3 laboratory of the Special Infectious Agents Unit, BSL-3 of King Fahd Medical Research Center at King Abdulaziz University in Jeddah, Saudi Arabia. Plaque reduction neutralization assays were performed to evaluate the neutralizing activity of anti-A29 IgY antibodies against Mpox virus. Serial dilutions of IgY antibodies were incubated with an equal volume of 0.01 MOI Mpox virus at 37° C. for 30-60 min. Subsequently, 200 μLs of the incubated mixture were added to 95-100% confluent Vero E6 cells in 12-well plates and incubated at 37° C. in 5% CO2 for 1 h, gently rocking every 10 minutes. Each assay included a cell control (PBS and cells) and a virus control (virus and cells). After incubation, the Vero cells were covered with an agarose-containing overlay medium of 1.5 mL to control the indiscriminate spreading of the virus. Plates were incubated for 72 h at 37° C. in a 5% carbon dioxide atmosphere. Vero cells were fixed with 10% formalin in phosphate-buffered saline followed by staining with 1% crystal violet in 50% ethanol.

The 50% neutralization concentration (NC₅₀) of Mpox-specific IgY was determined via the Reed-Muench method. The log IgY concentration was plotted against the percentage of inhibition of each concentration and the NC₅₀ was calculated following a nonlinear variable slope equation according to the equation: Y=100/(1+10\hat{ }((LogNC₅₀−X)×HillSlope)).

### Results

### Isolation and Purification of IgY

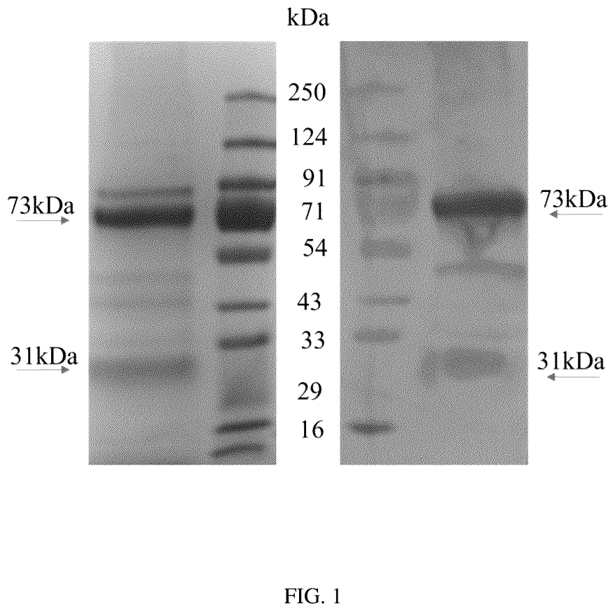

The measured concentration of the purified IgY was ˜17 mg/ml giving a total of 255 mg per one-egg yolk (≈15 mL). SDS-PAGE showed the dissociation of the IgY into ˜73 kDa and ˜31 kDa representing its heavy and light chain, respectively with a purity of 89% (FIG. 1).

### Reactivity of Anti-Mpox-A29 IgY Antibodies in the Chickens' Egg Yolks

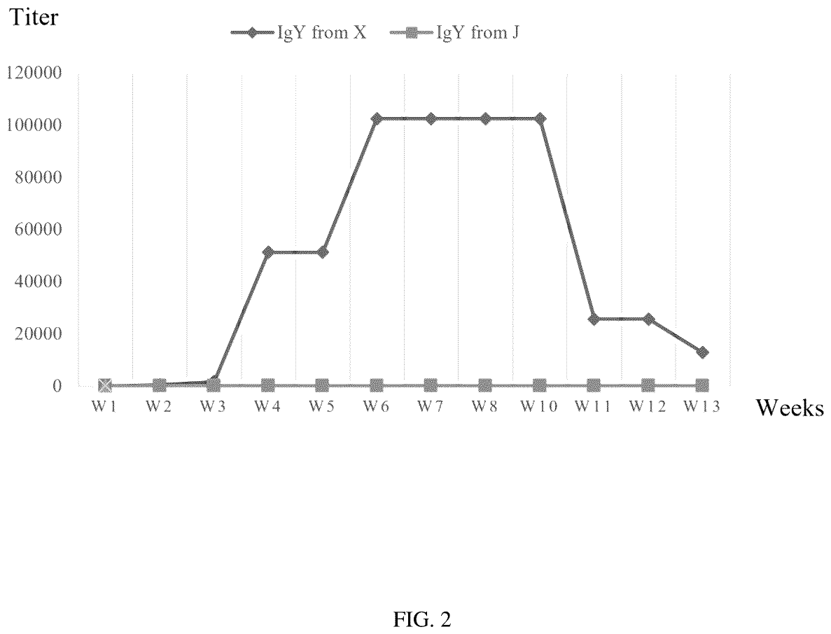

ELISA assay showed that the titer of Anti-Mpox-A29 IgYs in the eggs of the immunized group (X) started to rise at week 1 with a rising titer at W3 (1600) after the first booster dose and continued to rise (51200) at W5 (after second booster dose). The titer reached a maximum titer of 102400 at W6 and continued to W10. The IgY titer started declining at W11 (25600) to W13 (12800). IgY purified from the non-immunized group (J) did not show reactivity to the Mpox-A29 antigen (FIG. 2).

### Immunoreactivity of Anti-A29 IgY of the Mpox

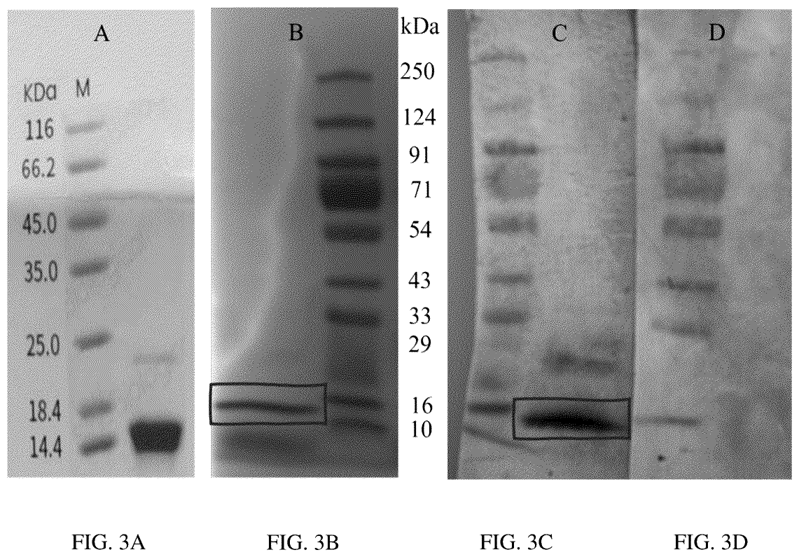

The specificity of anti-Mpox-A29 IgY antibodies were tested using Western blotting analysis (FIGS. 3A-D). IgY antibodies induced by the recombinant A29 protein immunization recognized the A29 protein at approximately 14 kDa (FIG. 3C) while nonimmunized hens (J) showed no bands at the same molecular weight.

### Cytotoxicity of Anti-Mpox-A29 IgY

Anti-Mpox-A29 IgY antibodies were tested for their cytotoxic effect on the Vero-E6 cells using a 10 mg/ml concentration range to 1 μg/ml. The IgY antibodies did not show any cytotoxic effect on the Vero cells in the concentration range investigated, thus the CC₅₀ of the anti-Mpox-A29 IgY antibodies is >10 mg/ml.

### REFERENCES

[1] “2022-23 Mpox (Monkeypox) Outbreak: Global Trends.” worldhealthorg.shinyapps.io/mpx_global/#5_Genomic_epidemiology (accessed Apr. 2, 2023).
[2] I. V. Babkin, I. N. Babkina, and N. V. Tikunova, “An Update of Orthopoxvirus Molecular Evolution,” Viruses, vol. 14, no. 2, pp. 1-13, 2022, doi: 10.3390/v14020388.
[3] R. M. Salcedo and M. G. Madariaga, “Monkeypox (hMPOX Infection): A Practical Review,” American Journal of Medicine, vol. 136, no. 3. Elsevier Inc., pp. 234-243, Mar. 1, 2023, doi: 10.1016/j.amjmed.2022.10.023.
[4] S. S. Marennikova, “Poxviruses isolated from clinically ill and asymptomatically infected monkeys and a chimpanzee,” Bull. Org. mond. St., vol. 46, pp. 613-620, 1972.
[5] Y. M. Dou, H. Yuan, and H. W. Tian, “Monkeypox virus: past and present,” World Journal of Pediatrics, vol. 19, no. 3. Zhejiang University School of Medicine Children's Hospital, pp. 224-230, Mar. 1, 2023, doi: 10.1007/s12519-022-00618-1.
[6] “Treatment Information for Healthcare Professionals |Mpox|Poxvirus|CDC.” cdc.gov/poxvirus/mpox/clinicians/treatment.html (accessed Apr. 2, 2023).
[7] H. Marcotte and L. Hammarström, “Passive Immunization: Toward Magic Bullets,” in Mucosal Immunology: Fourth Edition, vol. 2-2, Elsevier Inc., 2015, pp. 1403-1434.
[8] S. A. El-Kafrawy et al., “IgY antibodies: The promising potential to overcome antibiotic resistance,” Frontiers in Immunology, vol. 14. Frontiers Media S. A., Jan. 20, 2023, doi: 10.3389/fimmu.2023.1065353.
[9] D. León-Núñez, M. F. Vizcaíno-López, M. Escorcia, D. Correa, E. Pérez-Hernández, and F. Gómez-Chávez, “IgY Antibodies as Biotherapeutics in Biomedicine,” Antibodies, vol. 11, no. 4. MDPI, Dec. 1, 2022, doi: 10.3390/antib11040062.
[10] L. Lee, K. Samardzic, M. Wallach, L. R. Frumkin, and D. Mochly-Rosen, “Immunoglobulin Y for Potential Diagnostic and Therapeutic Applications in Infectious Diseases,” Frontiers in Immunology, vol. 12. Frontiers Media S. A., Jun. 9, 2021, doi: 10.3389/fimmu.2021.696003.
[11] A. D. Otter et al., “Analogous humoral antigen recognition between Monkeypox-infected and Smallpox-vaccinated individuals,” doi: 10.1101/2022.12.22.22283648.
[12] G. C. Carter, M. Law, M. Hollinshead, and G. L. Smith, “Entry of the vaccinia virus intracellular mature virion and its interactions with glycosaminoglycans,” J. Gen. Virol., vol. 86, no. 5, pp. 1279-1290, May 2005, doi: 10.1099/vir.0.80831-0.
[13] L. J. Hughes et al., “A highly specific monoclonal antibody against monkeypox virus detects the heparin binding domain of A27,” Virology, vol. 464-465, no. 1, pp. 264-273, 2014, doi: 10.1016/j.virol.2014.06.039.
[14] A. Polson, M. B. von Wechmar, and M. H. V. van Regenmortel, “Isolation of viral IgY antibodies from yolks of immunized hens.,” Immunol. Commun., vol. 9, no. 5, pp. 475-493, January 1980, doi: 10.3109/08820138009066010.

While the invention has been described in terms of its several exemplary embodiments, those skilled in the art will recognize that the invention can be practiced with modification within the spirit and scope of the appended claims. Accordingly, the present invention should not be limited to the embodiments as described above but should further include all modifications and equivalents thereof within the spirit and scope of the description provided herein.

1. A method of producing immunoglobulin Y (IgY) antibodies against monkeypox virus, comprising:
a) immunizing hens with a peptide comprising at least one immunogenic domain of A29 fusion protein from the monkeypox virus, and
b) isolating the IgY antibodies against the A29 fusion protein from yolks of eggs laid by the immunized hens.

2. The method of claim 1, wherein the peptide has the amino acid identity of SEQ ID NO:1.

3. The method of claim 1, wherein the peptide has the amino acid identity of Arg1-Ala125 of SEQ ID NO:1.

4. A pharmaceutical composition comprising immunoglobulin Y (IgY) antibodies against monkeypox virus, wherein the IgY antibodies are produced in eggs laid by hens immunized with a peptide comprising at least one immunogenic domain of A29 fusion protein from the monkeypox virus.

5. The pharmaceutical composition of claim 3, wherein the peptide has the amino acid sequence identity of SEQ ID NO:1.

6. A method of inhibiting or treating a monkeypox virus infection in a subject in need thereof, comprising the steps of:
a) isolating IgY antibodies to monkeypox virus, wherein said IgY antibodies to monkeypox virus are generated by immunizing hens with a peptide comprising at least one immunogenic domain of A29 fusion protein from the monkeypox virus;
b) preparing a pharmaceutically acceptable composition comprising the IgY antibodies; and
c) administering a therapeutically effective amount of the pharmaceutically acceptable composition to the subject.

7. The method of claim 6, wherein the peptide has the amino acid sequence identity of SEQ ID NO:1.

8. The method of claim 6, wherein the composition is administered via a route of administration selected from the group consisting of intravenous injection, intravenous infusion, intraperitoneal injection, intraperitoneal infusion, intranasal, topical, and oral.

9. The method of claim 5, wherein the therapeutically effective amount ranges from 0.1 to 1,000 mg/kg.

10. The method of claim 6, wherein the subject is a human.

11-12. (canceled)
